(19) United States
(12) Patent Application Publication
(10) Pub. No.: US 2005/0089915 A1
(43) Pub. Date: Apr. 28, 2005

(54) Hybridization-based fluorescence assay

(75) Inventors: Ekambar R. Kandimalla, Southboro, MA (US); Sudhir Agrawal, Shrewsbury, MA (US)

Correspondence Address: JOSEPH C. ZUCCHERO;KEOWN & ASSOCIATES, SUITE 1200, 500 WEST CUMMINGS PARK, WOBURN, MA, 01801, US

(73) Assignee: Hybridon, Inc., Cambridge, MA (US)

(21) Appl. No.: 10/974,129

(22) Filed: Oct. 27, 2004

Related U.S. Application Data
(60) Provisional application No. 60/514,667, filed on Oct. 27, 2003.

(51) Int. Cl. C12Q 1/68

(52) U.S. Cl. 435/6

(57) ABSTRACT

The invention provides a method for detecting the presence or absence of an oligonucleotide in a sample. In the method according to the invention, a sample is obtained that may contain a target oligonucleotide. An oligonucleotide that is complementary to the target oligonucleotide and a fluorophore are added to the sample to form a mixture. The mixture is then incubated under hybridization conditions to allow binding of the complementary oligonucleotide to the target oligonucleotide to form a duplex. The fluorophore binds to the duplex; and fluorescence is then detected by standard procedures. The detection of fluorescence is indicative of the presence of the target oligonucleotide in the sample.

### RELATED APPLICATION

This Application claims the benefit of U.S. Provisional Application Ser. No. 60/514,667, filed on Oct. 27, 2003, the entirety of which is incorporated herein by reference.

### BACKGROUND OF THE INVENTION

1. Field of the Invention

The invention relates to the detection and quantitation of specific nucleic acid sequences. More particularly, the invention relates to the detection and quantitation of target oligonucleotides in a sample.

2. Summary of the Related Art

Oligonucleotides have become indispensable tools in modern molecular biology, being used in a wide variety of techniques, ranging from diagnostic probing methods to PCR to antisense inhibition of gene expression and immunotherapy applications. This widespread use of oligonucleotides has led to an increasing demand for rapid, inexpensive and efficient methods for synthesizing oligonucleotides. Recently, considerable interest has been generated in the development of synthetic oligonucleotides as therapeutic or gene expression modulating agents in the antisense approach. For example, Agrawal, Trends in Biotechnology 10:152-158 (1992) extensively reviews the development of antisense therapeutic approaches. For an antisense therapeutic approach to be effective, oligonucleotides must be introduced into a patient and must reach the specific tissues to be treated. Consequently, there is a need to be able to detect the presence or absence oligonucleotides in a sample obtained from such tissues. Unfortunately, the various techniques for detecting specific unlabelled nucleic acid sequences present in a sample has been extended to polynucleotides, such as large DNA or RNA molecules. Current methods, such as UV absorbance, HPLC, capillary gel electrophoresis (CGE), PAGE, ELISA, and hybridization to immobilized probes are slower and/or less sensitive than desired. Moreover, these methods typically use fluorophores such as cyanines, ethidium bromide or Hoechst 33258 for quantitation of duplexes in solution as the flourophores bind to double-stranded DNA (ds DNA) and not single-stranded DNA (ss DNA). Temsamani et al., U.S. Pat. No. 5,558,992, teach a method for detecting specific synthetic oligonucleotides that are present in body fluid or tissue samples, however, this method is not sensitive enough to detect a small amount of oligonucleotide in a sample.

Due to the small size of oligonucleotides, special problems relating to nonspecific binding or background, as well as to absence of binding, nondetection or false negatives exist. Thus, there remains a need to develop more rapid and sensitive procedures for the detection and quantitation of specific synthetic oligonucleotide sequences present in a sample. Additionally, assays of ss DNA would also be useful in various molecular biology and diagnostics applications.

### BRIEF SUMMARY OF THE INVENTION

The invention provides a method for detecting the presence or absence of an oligonucleotide in a sample. In the method according to the invention, a sample is obtained that may contain a target oligonucleotide. An oligonucleotide that is complementary to the target oligonucleotide and a fluorophore are added to the sample to form a mixture. The mixture is incubated under hybridization conditions to allow binding of the complementary oligonucleotide to the target oligonucleotide to form a duplex. The fluorophore binds to the duplex; and fluorescence is then detected by standard procedures. The detection of fluorescence is indicative of the presence of the target oligonucleotide in the sample.

Another aspect of the invention provides a method for determining the concentration of an oligonucleotide present in a sample. In the method according to the invention, a sample is obtained that may contain a target oligonucleotide. An oligonucleotide that is complementary to the target oligonucleotide and a fluorophore are added to the sample to form a mixture. The mixture is then incubated under hybridization conditions to allow binding of the complementary oligonucleotide to the target oligonucleotide to form a duplex. The fluorophore binds to the duplex; and fluorescence is then detected by standard procedures. The measure of the fluorescence corresponds to the amount of duplex formed, which corresponds to the concentration of the target oligonucleotide in the sample.

Another aspect of the invention provides a method for detecting the presence or absence of two or more oligonucleotides in a sample. In the method according to the invention, a sample is obtained that may contain the two or more target oligonucleotides. Oligonucleotides that are complementary to the two or more target oligonucleotides are conjugated to different and distinct fluorophores. The complementary oligonucleotide-fluorophore conjugates are added to the sample to form a mixture. The mixture is then incubated under hybridization conditions to allow binding of the complementary oligonucleotide-fluorophore conjugates to the two or more target oligonucleotides to form a duplex(es). Fluorescence of one of the complementary oligonucleotide-fluorophore conjugates is then detected by standard procedures. The detection of fluorescence is indicative of the formation of a complementary oligonucleotide-fluorophore conjugate/target oligonucleotide duplex and, thus, the presence of one of the target oligonucleotides. The detection of fluorescence for the remaining complementary oligonucleotide-fluorophore conjugate(s) is then determined.

Another aspect of the invention provides a method for determining the concentration of two or more oligonucleotides in a sample. In the method according to the invention, a sample is obtained that may contain the two or more target oligonucleotides. Oligonucleotides that are complementary to the two or more target oligonucleotides are conjugated to different and distinct fluorophores. The complementary oligonucleotide-fluorophore conjugates are added to the sample to form a mixture. The mixture is then incubated under hybridization conditions to allow binding of the complementary oligonucleotide-fluorophore conjugates to the two or more target oligonucleotides to form a duplex(es). Fluorescence of one of the complementary oligonucleotide-fluorophore conjugates is then detected by standard procedures. The detection of fluorescence is indicative of the formation of a complementary oligonucleotide-fluorophore conjugate/target oligonucleotide duplex and, thus, the presence of one of the target oligonucleotides. The level of fluorescence is then measured and the level of fluorescence corresponds to the concentration of the two or more oligonucleotides in the sample. The detection and level of fluorescence for the remaining complementary oligonucleotide-fluorophore conjugate(s) is then determined.

### BRIEF DESCRIPTION OF THE DRAWINGS

FIG. 1. Schematic representation of an embodiment of the assay according to the invention. Reagent mixture containing ss oligonucleotide (complementary strand), which is complementary to the oligonucleotide in the test solution (known sequence), and fluorophore itself does not have fluorescence. When it is mixed with the test solution, i) a duplex formation between the test oligonucleotide and its complementary strand in the reagent mixture occurs, and ii) the fluorophore interacts with the in situ formed duplex giving fluorescence. The measure of the fluorescence corresponds to the amount of duplex formed, which corresponds to concentration of the test oligonucleotide in solution.

FIG. 2. Concentration-dependent fluorescence of the ethidium bromide-duplex complex of oligonucleotide 2 (oligo 2) in solution. Each value is an average of three independent readings±standard deviation. The fluorescence in the presence of control oligo 4 that does not have complementarity with oligo 1 is shown. Inset, plus signs are the fluorescence values obtained with a mixture of oligo 2 and oligo 4. Oligo 2 standard curve is shown for comparison.

FIG. 3. Concentration-dependent fluorescence of Hoechst 33258-oligo 2-duplex complex in solution. Each value is an average of three independent readings±standard deviation. The fluorescence in the presence of control oligo 4 that does not have complementarity with oligo 1 is shown.

FIG. 4. Concentration-dependent fluorescence of the ethidium bromide-duplex complex of phosphorothioate oligo 3 in solution. Each value is an average of three independent readings±standard deviation. Fluorescence curve of the ethidium bromide-duplex complex of phosphodiester oligo 2 is also shown for comparison.

### DETAILED DESCRIPTION OF THE PREFERRED EMBODIMENTS

The invention provides a method of detecting a target oligonucleotide(s) in a sample. As used herein, “oligonucleotides” include, but are not limited to, all polymers of 5′ to 3′ linked ribonucleosides, 2′-modified ribonucleosides and/or deoxyribonucleosides wherein the linkage may be a natural phosphodiester linkage or an artificial linkage, including without limitation a phosphorothioate, phosphorodithioate, phosphoramidate, alkylphosphonate, akylphosphonothioate, sulfonate, carbamate or phosphotriester linkage. Moreover, such oligonucleotides encompass oligonucleotides having modifications on the bases and/or sugar residues as well as those having nuclease resistance-conferring bulky substituents at the 3′ and/or 5′ end.

In some embodiments, the oligonucleotides each have from about 3 to about 50 nucleoside residues, preferably from about 13 to about 35 nucleoside residues, more preferably from about 15 to about 26 nucleoside residues. In some embodiments, the oligonucleotides have from about 5 to about 18, or from about 5 to about 14, nucleoside residues. As used herein, the term “about” implies that the exact number is not critical. Thus, the number of nucleoside residues in the oligonucleotides is not critical, and oligonucleotides having one or two fewer nucleoside residues, or from one to several additional nucleoside residues are contemplated as equivalents of each of the embodiments described above. In some embodiments, one or more of the oligonucleotides have 20 nucleotides.

As used herein, “sample” includes, but is not limited to, a solution, bodily fluid or tissue that may contain the target oligonucleotide(s). As used herein, “bodily fluid” includes, but is not limited to, blood, urine, sweat, mucous secretions, cerebrospinal fluid and synovial fluid. “Tissues” include those constituting any organ, such as lymphoid tissue, liver, kidney, lung, brain, intestine, smooth muscle, cardiac muscle, striated muscle, dermis and epidermis, among others. A method of detecting synthetic oligonucleotides extracted from bodily fluids or tissues is described in U.S. Pat. No. 5,558,992, which is incorporated herein by reference in its entirety.

The invention provides a method for detecting the presence or absence of an oligonucleotide present in a sample. In the method according to the invention, a sample is obtained that may contain a target oligonucleotide. An oligonucleotide that is complementary to the target oligonucleotide and a fluorophore are added to the sample to form a mixture. The mixture is then incubated under hybridization conditions to allow binding of the complementary oligonucleotide to the target oligonucleotide to form a duplex. The fluorophore binds to the duplex; and fluorescence is then detected by standard procedures. The detection of fluorescence is indicative of the presence of the target oligonucleotide in the sample. The method can also be used to detect and determine the concentration of a specific, target oligonucleotide in a mixture of oligonucleotides of different sequences. Alternatively, the fluorophore may be directly conjugated to the complementary oligonucleotide.

A fluorophore or fluorescent dye absorbs incident light and in response emits light at a different wavelength, i.e., a fluorescence. The fluorophore to be used in the methods according to the invention include, but are not limited to, ethidium bromide, Hoechst 33258 and PicoGreen. Ethidium bromide interacts with ds DNA through intercalation while Hoechst 33258 binds in the minor groove of the ds DNA with some preference for AT base pairs. Both fluoresce upon binding to ds DNA but neither binds to ss DNA. The fluorophore to be used should not emit a fluorescence, above normal background levels, unless a duplex between the complementary oligonucleotide and the target oligonucleotide has been formed. This reduces the amount of background or the number of false positive results and allows for target oligonucleotides to be quantitated in the presence of other unrelated sequences. One skilled in the art would readily be able to determine which fluorophore dye or dyes are best suited to a particular application. Preferably, the fluorophore should intercalate between paired bases in the double-stranded oligonucleotide. Alternatively, the fluorophore could bind to the major and/or minor groove of the ds DNA. Such fluorophores may have a preference for A/T base pairs while others may have a preference for G/C base pairs.

The method can be carried out in conventional microtiter plates such as, but not limited to, 96-well microtiter plate or in any other container or on any surface capable of holding liquid samples and of being scanned by the appropriate detection device, for example a plate reader or microscope. As the method is applicable in multiwell microtiter plate format, it can be used for rapid determination of oligonucleotide concentrations in biological and clinical samples and should be insensitive to the presence of proteins. The method works equally well with phosphorothioate oligonucleotides that are used most commonly in antisense applications.

Occasionally a target oligonucleotide can be found in more than one sample. For example, a target oligonucleotide that has been administered to an animal (e.g., a mammal such as a human) might be detected in the lung and the liver but not the kidney. In a further aspect of the invention, the presence or absence of an oligonucleotide in two or more samples is detected. In one embodiment of this aspect, samples from various sources (e.g., blood, heart, lung, liver) are collected and the presence or absence of the target oligonucleotide is determined for each sample. In another embodiment, the samples are obtained and are placed into different containers or surfaces, including without limitation, the wells in a multiwell microtiter plate. In this embodiment, the presence or absence of the target oligonucleotide in the samples can be detected simultaneously.

Another aspect of the invention provides for a method for determining the concentration of an oligonucleotide present in a sample. In the method according to the invention, a sample is obtained that may contain a target oligonucleotide. An oligonucleotide that is complementary to the target oligonucleotide and a fluorophore are added to the sample to form a mixture. The mixture is then incubated under hybridization conditions to allow binding of the complementary oligonucleotide to the target oligonucleotide to form a duplex. The fluorophore binds to the duplex; and fluorescence is then detected by standard procedures. The measure of the fluorescence corresponds to the amount of duplex formed, which corresponds to the concentration of the target oligonucleotide in the sample. Alternatively, the fluorophore may be directly conjugated to the complementary oligonucleotide. The intensity of fluorescence may be compared with samples of various known concentrations of ds oligonucleotide.

Occasionally a single sample contains more than one target oligonucleotide. Another aspect of the invention provides for a method for detecting the presence or absence of two or more oligonucleotides in a sample. In the method according to the invention, a sample is obtained that may contain the two or more target oligonucleotides. Oligonucleotides that are complementary to the two or more target oligonucleotides are conjugated to different and distinct fluorophores. The complementary oligonucleotide-fluorophore conjugates are added to the sample to form a mixture. The mixture is then incubated under hybridization conditions to allow binding of the complementary oligonucleotide-fluorophore conjugates to the two or more target oligonucleotides to form a duplex(es). Fluorescence of one of the complementary oligonucleotide-fluorophore conjugates is then detected by standard procedures. The detection of fluorescence is indicative of the formation of a complementary oligonucleotide-fluorophore conjugate/target oligonucleotide duplex and, thus, the presence of one of the target oligonucleotides. The detection of fluorescence for the remaining complementary oligonucleotide-fluorophore conjugate(s) is then determined.

Another aspect of the invention provides for a method for determining the concentration of two or more oligonucleotides in a sample. In the method according to the invention, a sample is obtained that may contain the two or more target oligonucleotides. Oligonucleotides that are complementary to the two or more target oligonucleotides are conjugated to different and distinct fluorophore. The complementary oligonucleotide-fluorophore conjugates are added to the sample to form a mixture. The mixture is then incubated under hybridization conditions to allow binding of the complementary oligonucleotide-fluorophore conjugates to the two or more target oligonucleotides to form a duplex(es). Fluorescence of one of the complementary oligonucleotide-fluorophore conjugates is then detected by standard procedures. The detection of fluorescence is indicative of the formation of a complementary oligonucleotide-fluorophore conjugate/target oligonucleotide duplex and, thus, the presence of one of the target oligonucleotides. The level of fluorescence is then measured and the level of fluorescence corresponds to the concentration of the two or more oligonucleotides in the sample. The detection and level of fluorescence for the remaining complementary oligonucleotide-fluorophore conjugate(s) is then determined.

The following examples are intended to further illustrate certain preferred embodiments of the invention and are not limiting in nature.

### EXAMPLE 1

### Oligonucleotides and Synthesis

Oligonucleotides 1 (5′-GTGAGTGAGAACAGGTGTCA-3′; PO), 2 (5′-TGACACCTGTTCTCACTCAC-3′; PO), 3 (5′-TGACACCTGTTCTCACTCAC-3′; PS), and 4 (5′-GCGTGCCTCCTCACTGGC-3′; PO) were synthesized on a 1 μmole scale using β-cyanoethylphosphoramidite chemistry on a PerSeptive Biosystems 8909 Expedite DNA synthesizer. PO and PS stand for phosphodiester and phosphorothioate backbones, respectely. The phosphoramidites required were obtained from PE Biosystems. Beaucage reagent was used as an oxidant to obtain the phosphorothioate backbone modification. All oligonucleotides were deprotected using standard protocols, purified by HPLC, and dialyzed against USP quality sterile water for irrigation (Braun). The oligos were lyophilized, dissolved again in distilled water and the concentrations were determined by UV absorbance at 260 nm.

### EXAMPLE 2

### Reagents

Both ethidium bromide and Hoechst 33258 were purchased from Aldrich-Sigma and the stock solutions were prepared in 10 mM disodium hydrogen phosphate buffer, pH 7.2 containing 150 mM NaCl and 2 mM MgCl₂.

### EXAMPLE 3

### Thermal Melting Studies

Thermal melting of duplexes involving the oligonucleotide of interest was measured in 1 mL 150 mM NaCl, 2 mM MgCl₂, and 10 mM disodium hydrogen phosphate, pH 7.2 buffer. The concentration of each oligonucleotide strand was 2.0 μM. Thermal melting measurements were carried out at 260 nm on a Perkin-Elmer Lambda 20 Spectrophotometer attached to a Peltier thermal controller and a personal computer using 1 cm path length quartz cuvettes at a heating rate of 0.5° C./min. Melting temperatures (Tₘ) were taken as the temperature of half-dissociation and were obtained from first derivative plots generated using the vendor supplied software.

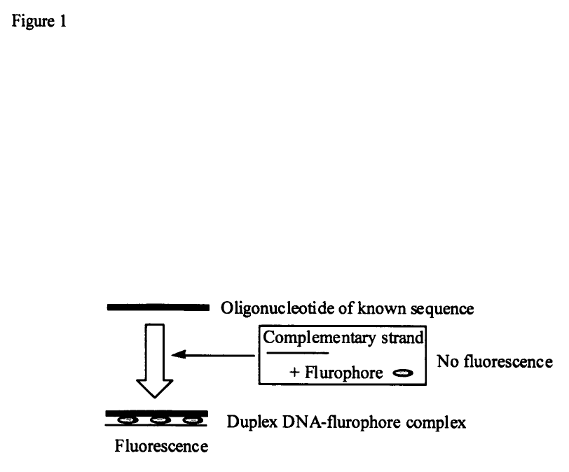

The assay is shown schematically in FIG. 1. Fluorophore and complementary oligonucleotide are added to the solution of oligonucleotide to be analysed. Following duplex formation and binding of fluorophore, fluorescence intensity gives the oligonucleotide concentration.

In the buffer used for the fluorescence assay, the duplex formed by complementary oligonucleotides 1 and 2 had a Tm of 66.4±0.75° C. while no duplex was detected with oligonucleotides 1 and 4 under the same conditions.

By varying concentrations of complementary oligo 1 and ethidium bromide, optimal values with minimal background fluorescence were determined to be between about 0.1 to 4.0 and 0.25 to 10.0 μM, respectively. Preferably the optimal values are 2.0 and 5.0 μM respectively. Similarly, optimal values of 1 and Hoechst 33258 were found to be between about 0.5 to 8.0 and 0.25 to 4.0 μM, respectively. Preferably the optimal values are 4.0 and 2.0 μM, respectively.

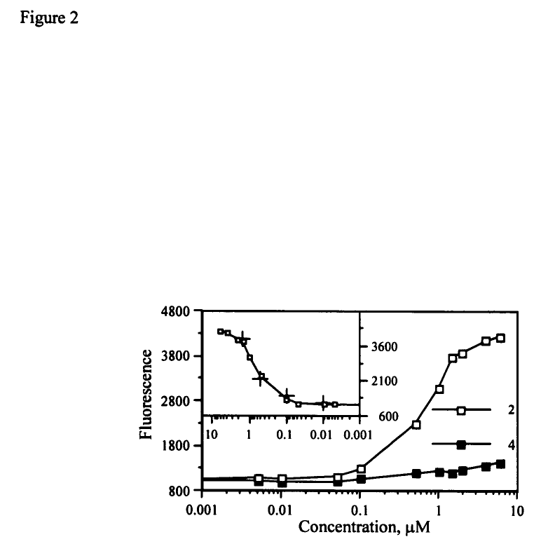

To examine specificity further, mixtures of different concentrations of oligos 2 and 4 in a 1:3 ratio were assayed using ethidium bromide. The results in FIG. 2 inset (plus signs) show that the presence of oligo 4 did not interfere with the determination of oligo 2.

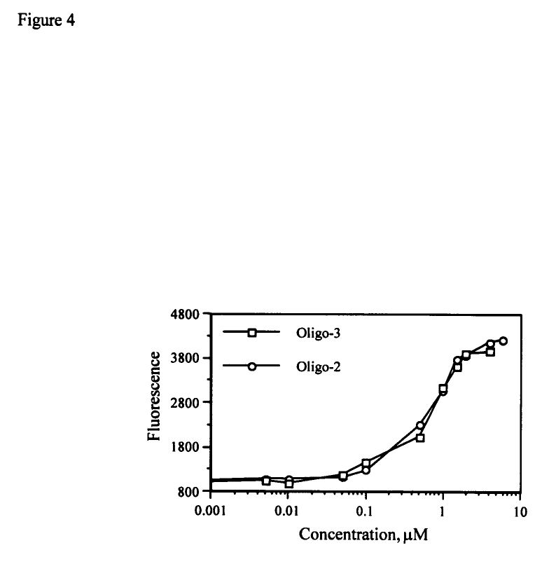

In general, phosphorothioate modified oligonucleotides are used for antisense applications in vivo because of their greater resistance to nucleases. To examine the influence of this modification on fluorescence, we used the phosphorothioate oligo 3 having the same sequence as oligo 2. The Tm of the duplex between oligos 1 and 3 was 57.2±1.0° C. in the buffer used for fluorescence assay. With ethidium bromide, oligo 3 produced fluorescence similar to the phosphodiester oligo 2 (FIG. 4). Similar results were obtained using Hoechst 33258 (data not shown). Thus, this assay is applicable to phosphorothioates as well as phosphodiester oligonucleotides.

Both ethidium bromide and Hoechst 33258 can be used to determine the concentrations of ss oligonucleotides by hybridization-induced fluorescence. The assay is sensitive and reproducible down to 60 ng/mL of a 20-mer ss oligonucleotide. The sensitivity of the reaction might be further increased with the use of other fluorophores such as, but not limited to, PicoGreen that give higher fluorescence yield upon binding to ds DNA.

### EXAMPLE 4

### Fluorescence Measurements

Fluorescence measurements were made on a Biotek Instruments Synergy HT Elx 808 Spectrofluorometer attached to a personal computer and using KC4 software. The excitation and emission monochromators were adjusted to a band width of 20 and 10 nm, respectively. The fluorescence was measured in a 96 well plate using 546 nm and 590 nm for ethidium bromide and 346 nm and 460 nm for Hoechst 33258 as excitation and emission wavelengths, respectively.

### EXAMPLE 5

### Standard Curve

All measurements were made in a 96-well plate in a final volume of 200-210 μL/well. The final concentrations of complementary strand and ethidium bromide in solution were 2.0 and 5.0 μM, respectively. Concentrations of oligonucleotide to be measured varied from 0.005 to 6.0 μM. The final concentrations of complementary strand and Hoechst 33258 in solution were 4.0 and 2.0 μM, respectively. All measurements were made in 150 mM NaCl, 2 mM MgCl₂, 10 mM sodium hydrogen phosphate, pH 7.2.

A typical standard curve generated for oligo 2 using ethidium bromide is shown in FIG. 2. Fluorescence increased linearly using 0.05 to 2.0 μM oligo 2 in a 200 μL solution. Under the same conditions, non-complementary control oligo 4 showed minimal fluorescence demonstrating the specificity of the reaction (FIG. 2). The lower limit of detection was about 0.01 μM of oligo 2 or 12 ng/200 μL of final assay solution.

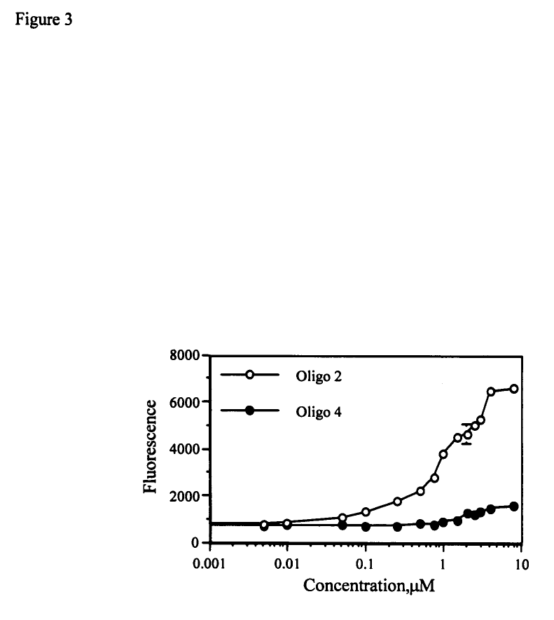

Similarly, the standard curve for oligo 2 with Hoechst 33258 is shown in FIG. 3. Fluorescence increased linearly using 0.01 to 4.0 μM oligo 2 while oligo 4 gave no appreciable fluorescence. Although Hoechst 33258 gave greater fluorescence, the lower limit of detection was the same as with ethidium bromide.

1. A method for detecting the presence or absence of an oligonucleotide in a sample, comprising the steps of:
a) obtaining a sample;
b) adding to the sample an oligonucleotide that is complementary to a target oligonucleotide to form a mixture;
c) adding a fluorophore to the mixture;
d) incubating the mixture under conditions to allow binding of the complementary oligonucleotide to the target oligonucleotide to form a duplex;
e) binding of the fluorophore to the duplex; and
f) detecting fluorescence being indicative of the presence of the target oligonucleotide in the sample.

2. The method of claim 1, wherein the sample is contained in a microtiter plate.

3. The method of claim 1, where the presence or absence or an oligonucleotide in two or more samples is detected.

4. The method of claim 3, wherein the two or more samples are contained in different wells of a multiwell microtiter plate.

5. The method of claim 1, wherein the complementary oligonucleotide is conjugated to the fluorophore prior to adding to the sample.

6. A method for determining the concentration of an oligonucleotide present in a sample, comprising the steps of:
a) Obtaining a sample;
b) Adding to the sample an oligonucleotide that is complementary to a target oligonucleotide to form a mixture;
c) Adding a fluorophore to the mixture;
d) Treating the mixture under conditions to allow binding of the complementary oligonucleotide to the target oligonucleotide to form a duplex;
e) Binding of the fluorophore to the duplex;
f) Detecting fluorescence being indicative of the presence of the target oligonucleotide in the sample; and
g) Measuring the level of fluorescence
wherein the level of fluorescence corresponds to the concentration of the target oligonucleotide in the sample.

7. The method of claim 6, wherein the sample is contained in a microtiter plate.

8. The method of claim 6, where the presence or absence or an oligonucleotide in two or more samples is detected.

9. The method of claim 8, wherein the two or more samples are contained in different wells of a multiwell microtiter plate.

10. The method of claim 6, wherein the complementary oligonucleotide is conjugated to the fluorophore prior to adding to the sample.

11. A method for detecting the presence or absence of two or more oligonucleotides in a sample, comprising the steps of:
a) Obtaining a sample;
b) Conjugating oligonucleotides that are complementary to two or more target oligonucleotides to different fluorphores;
c) Adding to the sample said complementary oligonucleotide-fluorophore conjugates to form a mixture;
d) Treating the mixture under conditions to allow binding of the complementary oligonucleotide-fluorophore conjugates to the two or more target oligonucleotides to form a duplex;
e) Detecting fluorescence of one of the complementary oligonucleotide-fluorophore conjugates being indicative of the presence of one of the target oligonucleotides; and
f) Repeating step e) for the remaining complementary oligonucleotide-fluorophore conjugate(s).

12. A method for determining the concentration of two or more oligonucleotides in a sample, comprising the steps of:
a) Obtaining a sample;
b) Conjugating oligonucleotides that are complementary to two or more target oligonucleotides to different fluorphores;
c) Adding to the sample said complementary oligonucleotide-fluorophore conjugates to form a mixture;
d) Treating the mixture under conditions to allow binding of the complementary oligonucleotide-fluorophore conjugates to the two or more target oligonucleotides to form a duplex;
e) Detecting fluorescence of one of the complementary oligonucleotide-fluorophore conjugates being indicative of the presence of one of the target oligonucleotides;
f) Measuring the level of fluorescence; and
g) Repeating steps e) and f) for the remaining complementary oligonucleotide-fluorophore conjugate(s);
wherein the level of fluorescence corresponds to the concentration of the two or more oligonucleotides in the sample.
